(19) United States
(12) Patent Application Publication
(10) Pub. No.: US 2024/0025029 A1
(43) Pub. Date: Jan. 25, 2024

(54) HOLDER FOR PERSONAL SAMPLE CONTAINER

(71) Applicant: P-STIK Medical Supplies Inc., Aldergrove (CA)

(72) Inventors: Daniel FLOKSTRA, Aldergrove (CA); Amanda KOOP, Abbotsford (CA)

(21) Appl. No.: 18/354,490

(22) Filed: Jul. 18, 2023

Related U.S. Application Data
(60) Provisional application No. 63/391,048, filed on Jul. 21, 2022.

(51) Int. Cl. B25J 1/02

(52) U.S. Cl. CPC B25J 1/02; A61B 10/0038

(57) ABSTRACT

Methods and apparatus for aiding in the collection of personal samples. An example personal sample holder apparatus may comprise first and second elongated arms pivotally coupled to one another by a pivot joint to permit relative pivotal movement between the first and second arms about a pivot axis. The personal sample holder apparatus may further comprise a sample collection vessel holder located at a sample-collection location of the second arm, the sample-collection location spaced apart from the pivot joint. The pivot joint may be lockable at a plurality of discrete locking configurations to prevent relative pivotal movement between the first and second arms under application of forces associated with personal sample collection. Each of the plurality of discrete locking configurations may be at a corresponding relative angular orientation between the first and second arms.

### REFERENCE TO RELATED APPLICATIONS

This application claims priority from, and for the purposes of the United States the benefit under 35 USC 119 in relation to, U.S. patent application No. 63/391048 filed 21 Jul. 2022, which is hereby incorporated herein by reference.

### FIELD

This invention relates to apparatus for collecting personal samples. Some embodiments provide holders for collecting personal samples (e.g. urine and/or stool). This invention may particularly have applications in the self-collection of personal samples.

### BACKGROUND

The collection of personal samples, such as urine and/or stool, can be required for a wide variety of reasons. By way of non-limiting example, the collection of personal samples is sometimes required to conduct some types of medical tests. Currently, the self-collection of personal samples typically involves strategically placing the appropriate collection vessel to receive urine and/or stool. This may prove to be challenging due to the required position of the collection vessel combined with one or both of reaching such positions and maintaining such positions during collection.

There is a general need and desire for apparatus for the collection of personal samples that aid in, ease and simplify the process of collecting personal samples.

### SUMMARY

This invention has a number of aspects. These include, without limitation:

Apparatus for the collection of personal samples; and

Methods for the collection of personal samples.

One aspect of the invention provides a personal sample holder apparatus for aiding in the collection of personal samples. The personal sample holder apparatus comprises: first and second elongated arms pivotally coupled to one another by a pivot joint to permit relative pivotal movement between the first and second arms about a pivot axis; a sample collection vessel holder located at a sample-collection location of the second arm, the sample-collection location spaced apart from the pivot joint; wherein the pivot joint is lockable at a plurality of discrete locking configurations to prevent relative pivotal movement between the first and second arms under application of forces associated with personal sample collection and each of the plurality of discrete locking configurations is at a corresponding relative angular orientation between the first and second arms.

The pivot joint may be configured to facilitate relative pivotal movement between locking configurations upon the application, to one or both of the first and second arms, of a force sufficient to deform one or both of the first and second arms.

The pivot joint may comprise a first plate. There may be physical interaction between the first plate and at least one of the first and second arms that locks the pivot joint in one of the locking configurations.

The first plate may comprise a plurality of teeth and a plurality of indents. The indents may provide concavities between the teeth. The teeth may protrude in a direction that has at least one directional component that is parallel with the pivot axis and the indents may open in a direction that has at least one directional component that is parallel with the pivot axis.

At least one angular dimension about the pivot axis of a first tooth may be different than that of a second tooth.

At least one angular dimension about the pivot axis of a first tooth and a second tooth may be the same.

At least one angular dimension about the pivot axis of a first indent may be different than that of a second indent.

At least one angular dimension about the pivot axis of a first indent and a second indent may be the same.

The teeth and indents may define discrete relative angular positions of the first and second arms about the pivot axis.

The first arm and the second arm may each comprise at least one sidewall that extends in a direction that has at least one directional component that is parallel to the pivot axis.

At least one of the sidewalls may project into one of the indents between two adjacent teeth to lock the pivot joint at one of the locking configurations.

At least one of the first and second arms may be deformable to move to an adjacent indent upon the application of a sufficient force to the first arm and/or the second arm.

The first plate may be integrally formed with one of the first and second arms.

The pivot joint may comprise a first plate and a second plate. Physical interaction between at least one of the first and second arms and the first and second plates may lock the pivot joint in one of the locking configurations.

The first plate may comprise a cylindrical bearing surface that extends in a direction that is parallel with the pivot axis. The pivot axis may extend through a cross-sectional center of the cylindrical bearing surface. The first play may comprise one or more connecting protrusions located at locations radially spaced apart from the pivot axis and angularly spaced apart around the pivot axis. The connecting protrusions may extend from the cylindrical bearing surface in directions that have at least a directional component that is parallel with the pivot axis.

The first plate may form a snap-together fitting with the second plate.

The first plate may comprises one or more resting protrusions located at locations radially spaced apart from the pivot axis, angularly spaced apart around the pivot axis and between pairs of connecting protrusions. The resting protrusions may extend from the cylindrical bearing surface in directions that have at least a directional component that is parallel with the pivot axis. The resting protrusions may extend, from the cylindrical bearing surface, for a shorter distance than the connecting protrusions.

The connecting protrusions may be integrally formed with the cylindrical bearing surface.

Distal ends of the connecting protrusions may be widened relative to shaft portions of the connecting protrusions.

The connecting protrusions may extend through openings on the second plate to secure the first plate to the second plate.

The openings may be radially spaced apart from the pivot axis and angularly spaced apart from one another around the pivot axis.

The second arm may comprise a bore-defining surface.

The cylindrical bearing surface may bear against the bore-defining surface to facilitate relative pivotal motion between the first and second arms about the pivot axis.

The first plate may be integrally formed with the first arm.

The second plate may be formed separately from the first and second arms.

The first and second plates may comprise a plurality of teeth and a plurality of indents. The indents may provide concavities between the teeth. Each tooth may protrude in a direction that has a directional component parallel with the pivot axis. Each indent may open in a direction that has a directional component parallel with the pivot axis.

At least one tooth of the first plate may have a corresponding tooth on the second plate.

At least one indent of the first plate may have a corresponding indent on the second plate.

At least one angular dimension about the pivot axis of a first tooth may be different than that of a second tooth.

At least one angular dimension about the pivot axis of a first tooth and a second tooth may be the same.

At least one angular dimension about the pivot axis of a first indent may be different than that of a second indent.

At least one angular dimension about the pivot axis of a first indent and a second indent may be the same.

The teeth and indents may define discrete relative angular positions for at least one of the first and second arms about the pivot axis.

The first arm and the second arm may each comprise at least one sidewall that extends in a direction that has at least one directional component that is parallel with the pivot axis.

At least one of the sidewalls may project into one of the indents of the first plate or the second plate between two adjacent teeth to lock the pivot joint at one of the locking configurations.

The at least one arm may be deformable to move to an adjacent indent upon the application of a sufficient force to the at least one arm.

The first arm or the second arm may comprise a handle.

The sample collection vessel holder may comprise a grip that comprises one or more raised portions.

The sample collection vessel holder or the second arm at the sample-collection location may comprise one or more snap-together fittings that allow the sample collection vessel holder to attach to the second arm.

Further aspects and example embodiments are illustrated in the accompanying drawings and/or described in the following description.

It is emphasized that the invention relates to all combinations of the above features, even if these are recited in different claims.

### BRIEF DESCRIPTION OF THE DRAWINGS

The accompanying drawings illustrate non-limiting example embodiments of the invention.

FIG. 1 is a perspective view of an example personal sample collector according to a particular embodiment. FIG. 1A is another perspective view of the FIG. 1 example personal sample collector.

FIG. 2 is a perspective view of an example handle arm of the FIG. 1 example personal sample collector.

FIG. 3 is a perspective view of an example sample-collection arm of the FIG. 1 example personal sample collector.

FIG. 4 is a perspective view of an example pivot joint plate of the FIG. 1 example personal sample collector. FIG. 4A is another perspective view of the FIG. 4 example pivot joint plate.

FIG. 5 is a perspective view of the FIG. 1 example personal sample collector in a collapsed configuration.

FIG. 6 is a perspective view of the FIG. 1 example personal sample collector in one particular extended configuration.

FIG. 7 is a perspective view of an example collection vessel holder of the FIG. 1 example personal sample collector.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

One aspect of the invention provides a bodily substance collection apparatus to be used, for example, to aid in the self-collection of urine or stool. The apparatus may comprise a first arm and a second arm. The second arm may be pivotally coupled to the first arm at a pivot joint for relative pivotal motion between the first and second arms about a pivot axis. The apparatus may further comprise a sample collection vessel holder located at, or attached to, an end of one of the first and second arms.

The pivot joint may comprise first and/or second plates positioned at the ends of the first and second arms. The first and second plates may each comprise teeth on a face of the plate, which may interact with (e.g. contact) the second and first arms to maintain the relative angular orientation between the first and second arms and, in the absence of sufficient external force, prevent relative pivotal motion between the first and second arms during personal sample collection.

The first and the second arms may each comprise a sidewall that extends transversely from the first or second arm. The sidewalls of the arms may engage with the teeth of the pivot joint plates.

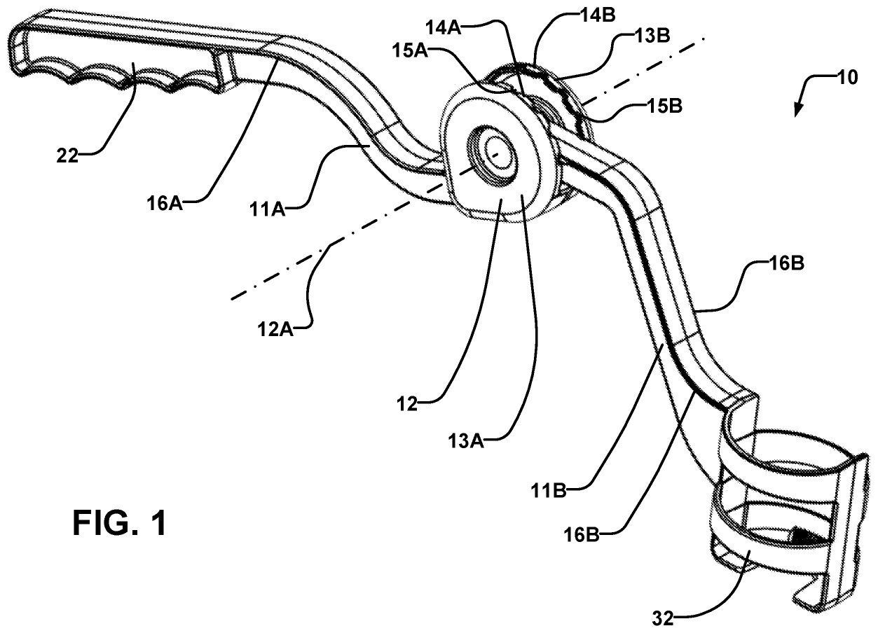

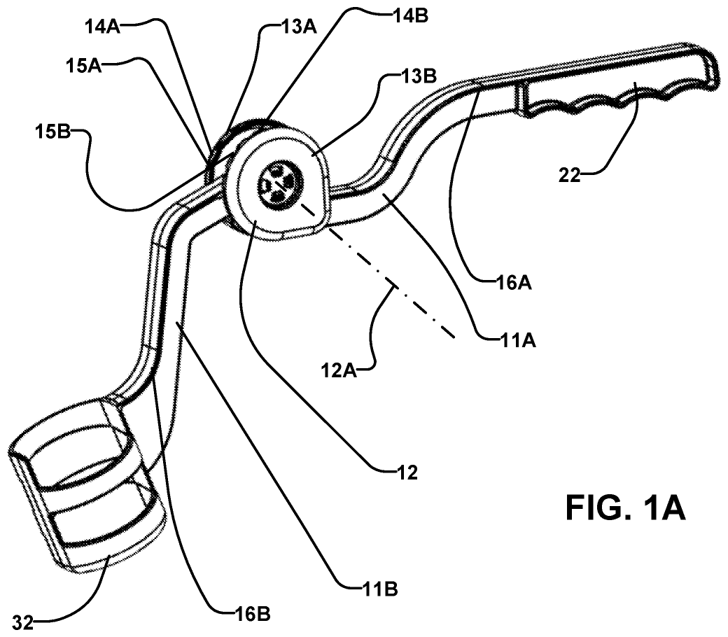

FIGS. 1 and 1A depict a personal sample holder apparatus 10 according to a particular example embodiment. Personal sample holder apparatus 10 may have applications in aiding in the collection of personal samples (e.g. urine, stool, etc.), particularly in the self-collection of personal samples.

Personal sample holder apparatus 10 comprises a handle arm 11A and a sample-collection arm 11B (referred to collectively as arms 11 or generally individually as an arm 11). Arms 11 are pivotally coupled to one another at pivot joint 12. Arms 11 may pivot relative to one another around pivot axis 12A.

In the particular case of the illustrated embodiment, pivot joint 12 comprises a first plate 13A and a second plate 13B (collectively plates 13). First and second plates 13A, 13B of the illustrated embodiment are respectively located at or near the ends of handle and sample-collection arms 11A, 11B and portions of plates 13 may extend radially away from pivot axis 12A. In some embodiments, plates 13 may surround pivot axis 12A.

As will be explained in more detail below, in the case of the illustrated embodiment, first plate 13A is integrally formed with (i.e. is a part of) handle arm 11A and second plate 13B is separately embodied. In some embodiments, second plate 13B is integrally formed with (i.e. is a part of) sample-collection arm 11B and first plate 13A is separately embodied. In some embodiments, first and second plates 13A, 13B are integrally formed with handle and sample-collection arms 11A, 11B. In some embodiments, both of plates 13 may be separate parts from arms 11. In some embodiments, both of plates 13 may form a part of handle arm 11A, in which case both of plates 13 may interact with sample-collection arm 11B. In some embodiments, both of plates 13 may form part of sample-collection arm 11B.

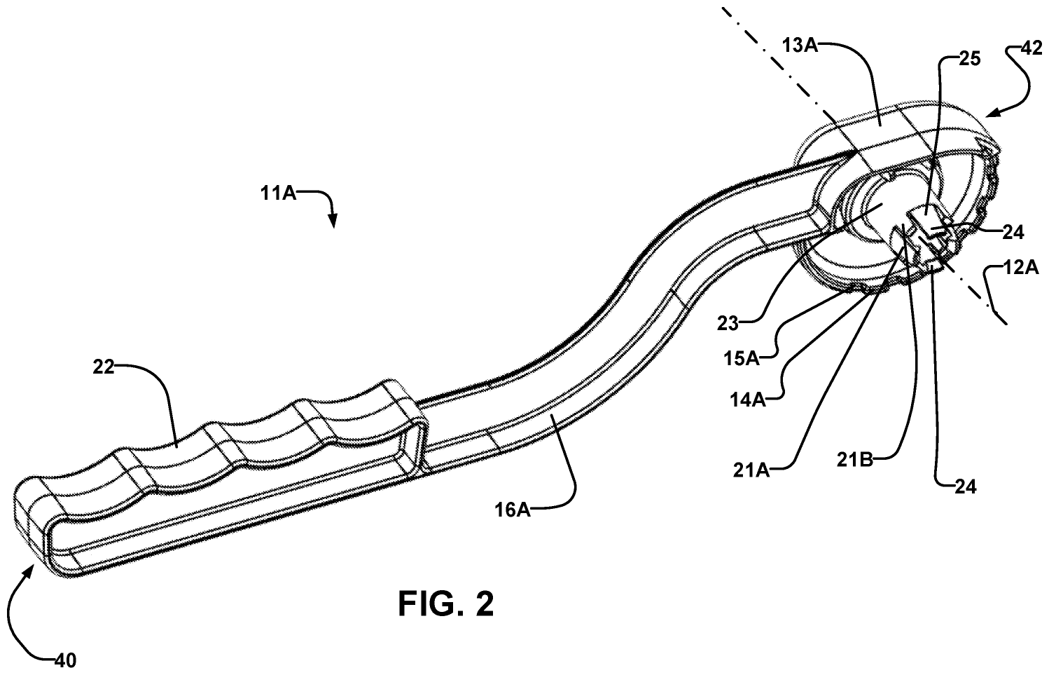

FIG. 2 depicts handle arm 11A of the FIG. 1 personal sample holder apparatus 10 according to a particular embodiment. Handle arm 11A may have a generally elongated body comprising a handle 22 located at or near one of its ends (handle end 40) and plate 13A at or near the opposing one of its ends (pivot end 42). Handle arm 11A may comprise cylindrical bearing surface 23 which extends in a direction that is parallel with pivot axis 12A. Pivot axis 12A may extend through a cross-sectional center of cylindrical bearing surface 23. Handle arm 11A may further comprise one or both of one or more connecting protrusions 21A and one or more resting protrusions 21B (individually or collectively protrusions 21). Protrusions 21 may be located at locations radially spaced apart from pivot axis 12A and may be angularly spaced apart around pivot axis 12A. At least one resting protrusion 21B may be circumferentially located between connecting protrusions 21A. Protrusions 21 may extend in directions that have at least a directional component that is parallel to pivot axis 12A. Resting protrusions 21B may extend for a shorter distance than connecting protrusions 21A in a direction that is parallel to pivot axis 12A. Protrusions 21 may be connected to, integrally formed with, or otherwise attached to cylindrical bearing surface 23. Protrusions 21 may extend from an edge of cylindrical bearing surface 23 that is most distal from first plate 13A.

Cylindrical bearing surface 23 and/or protrusions 21 may interact (e.g. connect) with one or both of sample-collection arm 11B and second plate 13B to connect handle arm 11A to sample-collection arm 11B and/or second plate 13B. Connecting protrusions 21A may comprise barbs or widened portions 24 at or near their distal ends to facilitate this interaction. Connecting protrusions 21A may also comprise shafts 25.

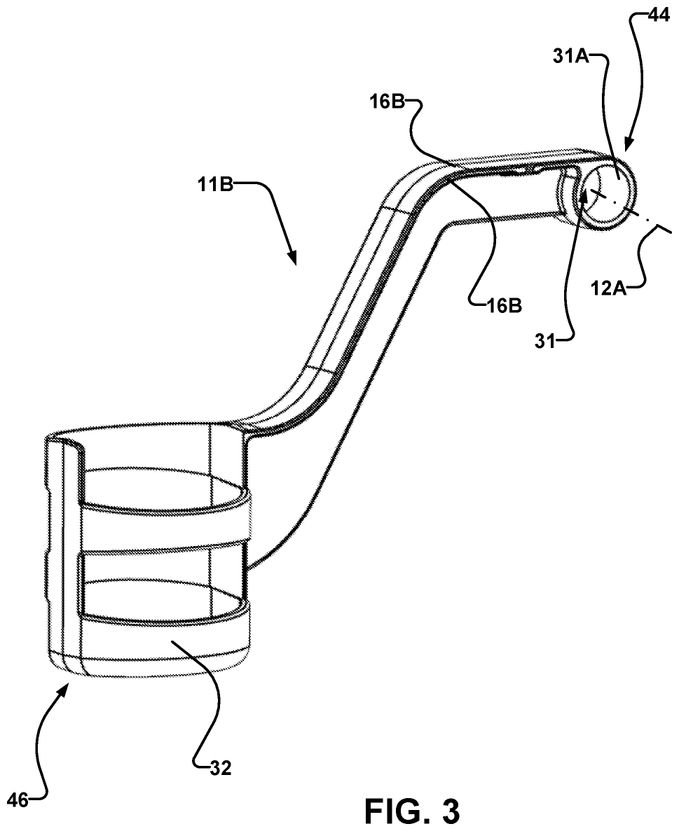

FIG. 3 depicts sample-collection arm 11B of the FIG. 1 personal sample holder apparatus 10 according to a particular embodiment. Sample-collection arm 11B may have a generally elongated body comprising a bore-defining surface 31A that defines a bore 31 located at or near one of its ends (pivot end 44) and holder 32 at or near the opposing one of its ends (holder end 46). Bore 31 of the illustrated embodiment is cylindrically shaped, extends in a direction that is parallel with pivot axis 12A and pivot axis 12A extends through a cross-sectional center of bore 31. In the particular case of the illustrated embodiment, at least a portion of a radially outwardly facing surface of one or more of cylindrical bearing surface 23 and protrusions 21 bears against bore-defining surface 31A of sample-collection arm 11B to facilitate relative rotational motion of arms 11A, 11B about pivot axis 12A. Connecting protrusions 21A of handle arm 11A (FIG. 2) may extend through bore 31 to engage with second plate 13B as described in more detail below.

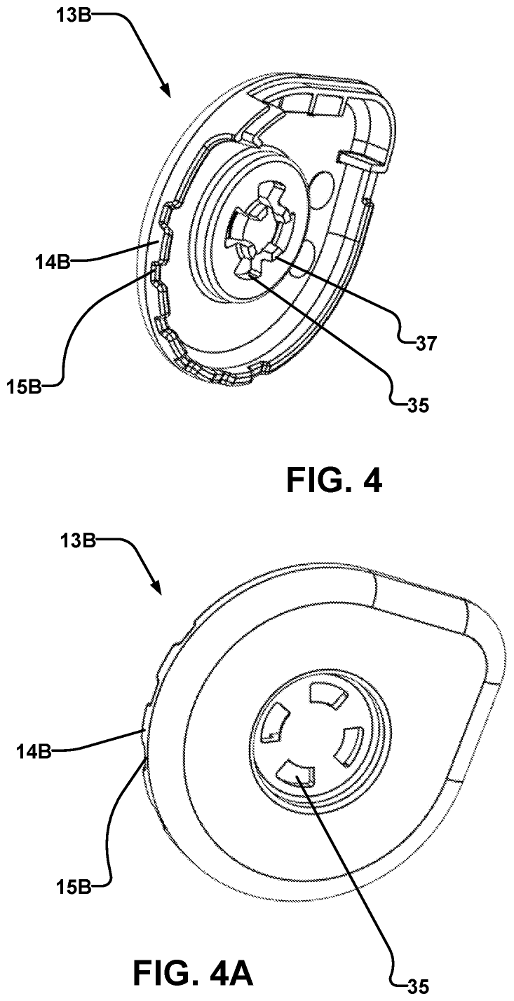

FIGS. 4 and 4A depict second plate 13B according to a particular embodiment. Second plate 13B may comprise one or more spacing protrusions 37 which extend in directions that are parallel with pivot axis 12A at locations that are radially spaced apart from pivot axis 12A and angularly spaced apart from one another around pivot axis 12A. Spacing protrusions 37 may comprise surfaces that come into contact with surfaces of resting protrusions 21B. Spacing protrusions 37 may bear against surfaces of resting protrusions 21B which may help maintain spacing between handle arm 11A and second plate 13B when handle arm 11A is coupled to second plate 13B.

Second plate 13B may be shaped to define notches 35 which are located at locations that are radially spaced apart from pivot axis 12A and angularly spaced apart from one another around pivot axis 12A. Connecting protrusions 21A of handle arm 11A (FIG. 2) may extend through notches 35, so that barbs or widened portions 24 of connecting protrusions 21A secure handle arm 11A to second plate 13B (with sample-collection arm 11B therebetween) and prevent second plate 13B from coming apart from handle arm 11A. In particular embodiments, this engagement between connecting protrusions 21A and second plate 13B is a resilient deformation “snap together” fitting, where connecting protrusions 21A are deformed to extend barbs or widened portions 24 through notches 35 and then, once projected through notches 35, restorative deformation forces (i.e. forces that tend to restore connecting protrusions 21A to their non-deformed states) tend to maintain the connection between barbs or widened portions 24 of connecting protrusions 21A and notches 35. In the illustrated embodiments, connecting protrusions 21A may be deformed in radial directions so that barbs or widened portions 24 move toward pivot axis 12A to make the “snap-together” fitting, and the associated restorative deformation forces may tend to force barbs or widened portions 24 in radial directions away from pivot axis 12A and into notches 35. In some embodiments, the directions of the original deformation and the restorative deformation forces may be reversed. In some embodiments, surfaces of shafts 25 of connecting protrusions 21A and/or the surfaces of barbs or widened portions 24 may be sized and/or shaped to facilitate deformation of connecting protrusions 21A as they are inserted into notches 35 and such that the restorative deformation forces tend to force second plate 13B toward handle arm 11A in a direction aligned with pivot axis 12A.

In the illustrated embodiments, first plate 13A (best seen in FIG. 2) and second plate 13B (best seen in FIGS. 4 and 4A) each respectively comprise teeth 14A and 14B (collectively teeth 14) and indents 15A and 15B (collectively indents 15). Teeth 14 may have shapes which protrude in directions that have at least a component oriented in directions parallel with pivot axis 12A. Indents 15 provide concavities (or spacing) between teeth 14 that open in directions that have at least a component oriented in directions parallel with pivot axis 12A. Teeth 14 may be angularly spaced apart from one another (by indents 15) about pivot axis 12A and may be located on a portion of the perimeter of plates 13. Some or all of teeth 14A, 14B may vary in size or be the same size. Some or all of indents 15A, 15B may vary in size or be the same size. Some or all of indents 15 may be trapezoidal in shape. Teeth 14A may align with teeth 14B such that each tooth 14A has a corresponding tooth 14B and each indent 15A has a corresponding indent 15B. Teeth 14A may align with indents 15B and teeth 14B may align with indents 15A, such that each tooth 14A has a corresponding indent 15B and each tooth 14B has a corresponding indent 15A.

The number of indents 15 and/or teeth 14 may vary between embodiments. In some embodiments, the number of teeth 14A and the number of teeth 14B may be between 3-10. In some embodiments, the number of indents 15A and the number of indents 15B may be between 3-10. The angular extension of teeth 14 and indents 15 around pivot axis 12A may vary between embodiments. In some embodiments, the combined angular extension of teeth 14A and indents 15A and/or teeth 14B and indents 15B around pivot axis 12A may be in a range of 0°-270°. In some embodiments, the combined angular extension of teeth 14A and indents 15A and/or teeth 14B and indents 15B around pivot axis 12A may be in a range of 90°-270°.

As shown best in FIG. 1, handle and sample-collection arms 11A, 11 B may respectively comprise sidewalls 16A, 16B (collectively, sidewalls 16) which extend in directions that have components that are parallel with pivot axis 12A and these sidewalls 16 may engage with teeth 14 by projecting into indents 15 between teeth 14, such that teeth 14 maintain the relative angular orientation between arms 11 and, in the absence of sufficient external force, prevent relative pivotal motion between arms 11 during sample collection. It should be understood that, in this disclosure, interaction of arms 11 (or sidewalls 16) with teeth 14 includes projection of sidewalls 16 into indents 15 between teeth 14. In the particular case of the illustrated embodiment, sidewalls 16B of sample-collection arm 11B project in both directions that are parallel with pivot axis 12A and engage teeth 14A (project into indents 15A) of first plate 13A and engage teeth 14B (project into indents 15B) of second plate 13B, while sidewalls 16A of handle arm 11A project in a direction that is parallel with pivot axis 12A toward second plate 13B to engage with teeth 14B (project into indents of second plate 13B. In some embodiments, sidewalls 16A of handle arm 11A may project in a direction that is parallel with pivot axis 12A toward second plate 13B and engage teeth 14B (project into indents 15B) of second plate 13B, while sidewalls of sample-collection arm 11B may project in a direction that is parallel with pivot axis 12A toward first plate 13A and engage teeth 14A (project into indents 15A) of first plate 13A.

Sidewalls 16 may remain within indents 15 unless or until a sufficient force (which is greater than that normally associated with personal sample collection) is applied to one or both of arms 11. In this way, indents 15 may maintain the relative angular orientation of arms 11 during personal sample collection. When a sufficient force is applied to an arm 11, such arm 11 may pivot relative to pivot axis 12A. The application of such a force may result in the elastic deformation of various components of apparatus 10 (e.g. arms 11, plates 13, sidewalls 16, teeth 14, protrusions 21A and/or the like). The elastic deformation may allow relative pivotal motion between handle and sample-collection arms 11A, 11B about pivot joint 12 (pivot axis 12A). When external force ceases to be applied to an arm 11, restorative forces may cause the corresponding sidewall 16 to project into a nearest indent 15, thereby locking the relative angular orientation of arms 11 relative to other another again.

A force may be considered to be sufficient if it is above a suitable threshold. The threshold may be equal to or greater than a force that may generally be applied to an arm 11 during the collection of a personal sample. Setting a threshold in this manner may ensure arms 11 maintain their positions within indents 15 (and that the relative angular orientation of arms 11 about pivot joint 12 is locked) during the collection of a personal sample. The threshold may vary at different relative angular orientations of arms 11. For example, the threshold may be relatively large when apparatus 10 is in a typical sample collection configuration and may be relatively low when apparatus 10 is in a storage configuration. The threshold may vary between embodiments. These thresholds may be designed by suitable sizing of one or both of one or more sidewalls 16 and one or more teeth 14 (or, correspondingly, the depth of one or more indents 15) and/or the materials from which personal sample holder apparatus 10 is constructed.

Teeth 14 and/or sidewalls 16 may comprise one or more beveled edges. Beveling may aid arms 11 pivot relative to pivot axis 12A. For example, beveling may reduce the force threshold required to be applied on an arm 11 for it to pivot relative to the other arm about pivot axis 12A. For example, beveling may aid guide sidewalls 16 into indents 15.

Indents 15 may be shaped and/or sized to maintain the angular position of a sidewall 16. To this effect, some or all of indents 15 may be sized to be approximately the thickness of a sidewall 16. In some embodiments, at least one indent 15 may be sized to fit two sidewalls 16 in which case the at least one indent 15 may be sized to be approximately the combined thickness of sidewalls 16.

Teeth 14 and indents 15 may define discrete relative angular positions (orientations) for arms 11 around pivot axis 12A. More specifically, in the case of the illustrated embodiment, the engagement of sidewalls 16B of second arm 11B with teeth 14A, 14B (projection of sidewalls 16B into indents 15A, 15B) defines discrete relative angular positions (orientations) for arms 11 about pivot axis 12A. The angular size and/or number of teeth 14 and indents 15 may define the relative angular spacing and/or number of discrete relative angular positions (orientations) for arms 11 about pivot axis 12A. The discrete relative angular positions for arms 11 may have regular angular intervals or these angular intervals may vary. For example, at least one angular dimension about pivot axis 12A may vary between two teeth 14 and/or two indents 15. For example, in an angular dimension about pivot axis 12A, as the angular dimension of a tooth 14 increases so does the angular interval between adjacent indents 15.

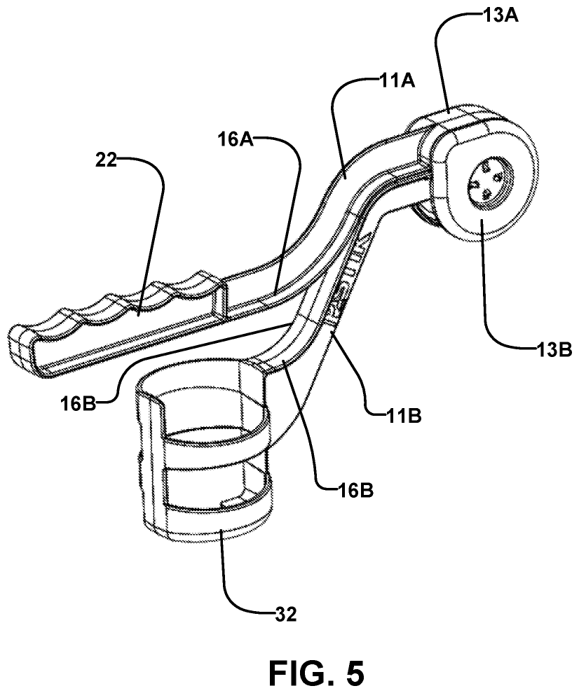

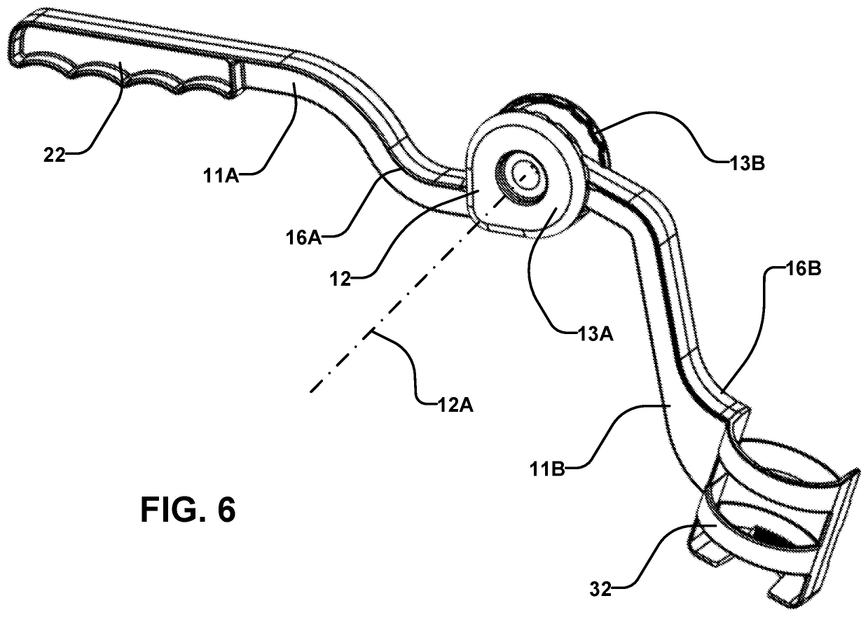

The relative angular range over which arms 11 may be pivoted may depend on the extension of teeth 14 and indents 15 around pivot axis 12A in handle arm 11A and/or second plate 13B. In some embodiments, this relative angular range may be in a range of 0°-270°. In some embodiments, this relative angular range may be in a range of 0°-180°. FIG. 5 shows personal sample holder apparatus 10 where arms 11 are at a relative angular orientation that is near 0°. This angular orientation may be convenient for storage and/or transport of personal sample holder apparatus 10. FIGS. 1 and 6 shows personal sample holder apparatus 10 in a configuration that may be convenient for self-collection of stool and/or urine samples.

As discussed above, connecting protrusions 21A (e.g. the surfaces of shafts 25 and/or barbs or widened portions 24) may be shaped such that restorative deformation forces associated with the “snap-together” connection between connecting protrusions 21A and notches 35 tend to pull second plate 13B toward first plate 13A (e.g. in directions parallel with pivot axis 12A). This same restorative deformation force may similarly force sidewalls 16 into engagement with teeth 14 (force sidewalls 16 to project into indents 15).

As discussed briefly above, handle arm 11A has an elongated and curved shape and comprises a handle 22 which is spaced apart from pivot joint 12. Handle 22 may provide a grip for personal sample holder apparatus 10. Handle 22 may advantageously improve the stability of personal sample holder apparatus 10 while in use by a user which in turn may make personal sample holder apparatus 10 easier to use.

As discussed briefly above, sample-collection arm 11B has an elongated and curved shape and comprises a holder 32 which is spaced apart from pivot joint 12. In some embodiments holder 32 may be attachable and detachable to sample-collection arm 11B. For example, holder 32 may be attached with suitable snap-together fittings located on one or both of holder 32 and sample-collection arm 11B. The ability to attach and detach holder 32 may allow various vessel holders to be substitutable as

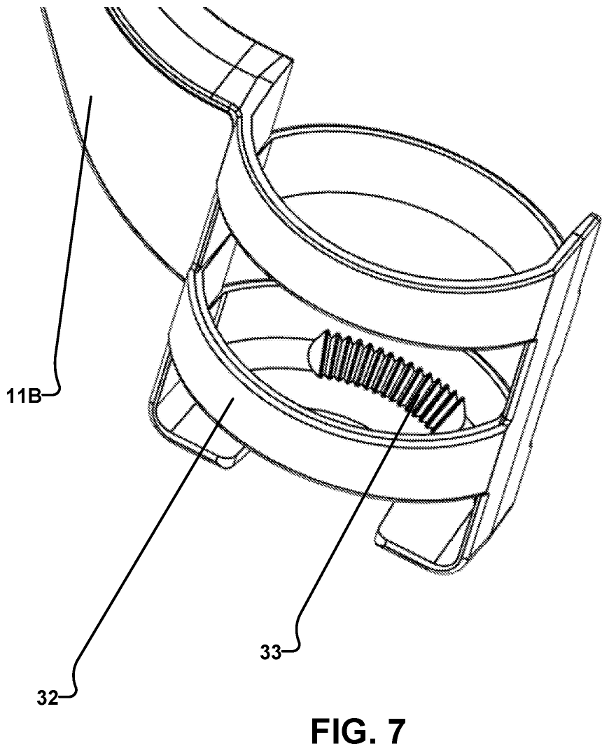

holder 32, for example vessel holders that vary in size. FIG. 7 depicts holder 32 in more detail. Holder 32 of the illustrated embodiment is a fixed size to firmly receive and hold a standard size collection vessel. Holder 32 may be of a shape that fits the outer shape profile of the collection vessel. For example, the collection vessel is typically cylindrical. In such cases holder 32 may be a hollow cylinder with an inner diameter that is slightly larger than an outer diameter of the collection vessel. Holder 32 may optionally comprise grip 33. Grip 33 may increase the friction between the collection vessel and holder 32. The increase in friction may help the collection vessel stay in holder 32. Grip 33 may comprise a portion of the base of holder 32. Grip 33 may comprise one or more raised portions and/or ridges. Grip 33 may be made of an elastomeric material, such as silicone and/or the like. In some embodiments, holder 32 may comprise a suitable closure mechanism that permits holder 32 to receive and hold collection vessels having different shape profiles. In some embodiments, sample-collection arm 11B may be configured with a suitable holder interchange mechanism that permits holders 32 of several sizes and/or shapes to be connected and/or disconnected from sample-collection arm 11B. The ability to swap such holders 32 may allow personal sample holder apparatus 10 to hold collection vessels having different shape profiles.

One or both of arms 11 may be curved. Curvature(s) in one or both of arms 11 may help position holder 32 and handle 22 in fixed positions from which it is easier to collect personal samples. Curvature(s) in one or both of arms 11 may aid in the storage of personal sample holder apparatus 10. For example, curvatures in arms 11 may help personal sample holder take up less space (see e.g. FIG. 5) when compared to arms 11 having straight shapes.

The components of personal sample holder apparatus 10 may be injection molded, press molded or otherwise fabricated and may be made out of any suitable material, such as hard plastic, metal and/or the like. Personal sample holder apparatus 10 may be made with one or more moulds. For example by injection moulding or pressed moulding.

The invention has a number of non-limiting aspects. Non-limiting aspects of the invention provide:

1. A personal sample holder apparatus for aiding in the collection of personal samples, the personal sample holder apparatus comprising:

first and second elongated arms pivotally coupled to one another by a pivot joint to permit relative pivotal movement between the first and second arms about a pivot axis;

a sample collection vessel holder located at a sample-collection location of the second arm, the sample-collection location spaced apart from the pivot joint;

wherein the pivot joint is lockable at a plurality of discrete locking configurations to prevent relative pivotal movement between the first and second arms under application of forces associated with personal sample collection and each of the plurality of discrete locking configurations is at a corresponding relative angular orientation between the first and second arms.

2. The personal sample holder according to aspect 1 wherein the pivot joint is configured to facilitate relative pivotal movement between locking configurations upon the application, to one or both of the first and second arms, of a force sufficient to deform one or both of the first and second arms.
3. The personal sample holder according to aspect 1 or 2 wherein the pivot joint comprises a first plate, wherein there is physical interaction between the first plate and at least one of the first and second arms that locks the pivot joint in one of the locking configurations.
4. The personal sample holder according to aspect 3 wherein the first plate comprises a plurality of teeth and a plurality of indents, wherein the indents provide concavities between the teeth, wherein the teeth protrude in a direction that has at least one directional component that is parallel with the pivot axis and the indents open in a direction that has at least one directional component that is parallel with the pivot axis.
5. The personal sample holder according to aspect 4 wherein at least one angular dimension about the pivot axis of a first tooth is different than that of a second tooth.
6. The personal sample holder according aspect 4 wherein at least one angular dimension about the pivot axis of a first tooth and a second tooth are the same.
7. The personal sample holder according to any one of aspects 4 to 6 wherein at least one angular dimension about the pivot axis of a first indent is different than that of a second indent.
8. The personal sample holder according to any one of aspects 4 to 7 wherein at least one angular dimension about the pivot axis of a first indent and a second indent are the same.
9. The personal sample holder according to any one of aspects 4 to 8 wherein the teeth and indents define discrete relative angular positions of the first and second arms about the pivot axis.
10. The personal sample holder according to any one of aspects 4 to 9 wherein the first arm and the second arm each comprise at least one sidewall that extends in a direction that has at least one directional component that is parallel to the pivot axis.
11. The personal sample holder according to aspect 10 wherein at least one of the sidewalls projects into one of the indents between two adjacent teeth to lock the pivot joint at one of the locking configurations.
12. The personal sample holder according to any one of aspects 4 to 11 wherein at least one of the first and second arms is deformable to move to an adjacent indent upon the application of a sufficient force to the first arm and/or the second arm.
13. The personal sample holder according to any one of aspects 2 to 12 wherein the first plate is integrally formed with one of the first and second arms.
14. The personal sample holder according to aspect 1 or 2 wherein the pivot joint comprises a first plate and a second plate, wherein physical interaction between at least one of the first and second arms and the first and second plates locks the pivot joint in one of the locking configurations.

15. The personal sample holder according to aspect 14 wherein the first plate forms a snap-together fitting with the second plate.

16. The personal sample holder according to aspect 15 or 16 wherein the first plate comprises:

a cylindrical bearing surface that extends in a direction that is parallel with the pivot axis, wherein the pivot axis extends through a cross-sectional center of the cylindrical bearing surface; and

one or more connecting protrusions located at locations radially spaced apart from the pivot axis and angularly spaced apart around the pivot axis, wherein the connecting protrusions extend from the cylindrical bearing surface in directions that have at least a directional component that is parallel with the pivot axis.

17. The personal sample holder according to aspect 16 wherein the first plate comprises:

one or more resting protrusions located at locations radially spaced apart from the pivot axis, angularly spaced apart around the pivot axis and between pairs of connecting protrusions, wherein the resting protrusions extend from the cylindrical bearing surface in directions that have at least a directional component that is parallel with the pivot axis
wherein the resting protrusions extend, from the cylindrical bearing surface, for a shorter distance than the connecting protrusions.
   18. The personal sample holder according to any one of aspects 16 and 17 wherein the connecting protrusions are integrally formed with the cylindrical bearing surface.
   19. The personal sample holder according to any one of aspects 16 to 18 wherein distal ends of the connecting protrusions are widened relative to shaft portions of the connecting protrusions.
   20. The personal sample holder according to any one of aspects 16 to 19 wherein the connecting protrusions extend through openings on the second plate to secure the first plate to the second plate.
   21. The personal sample holder according to aspect 20 wherein the openings are radially spaced apart from the pivot axis and angularly spaced apart from one another around the pivot axis.
   22. The personal sample holder according to any one of aspects 16 to 21 wherein the second arm comprises a bore-defining surface.
   23. The personal sample holder according to aspect 22 wherein the cylindrical bearing surface bears against the bore-defining surface to facilitate relative pivotal motion between the first and second arms about the pivot axis.
   24. The personal sample holder according to any one of aspects 14 to 23 wherein the first plate is integrally formed with the first arm.
   25. The personal sample holder according to any one of aspects 14 to 24 wherein the second plate is formed separately from the first and second arms.
   26. The personal sample holder according to any one of aspects 14 to 25 wherein the first and second plates comprise a plurality of teeth and a plurality of indents, wherein the indents provide concavities between the teeth, wherein each tooth protrudes in a direction that has a directional component parallel with the pivot axis and each indent opens in a direction that has a directional component parallel with the pivot axis.
   27. The personal sample holder according to aspect 26 wherein at least one tooth of the first plate has a corresponding tooth on the second plate.
   28. The personal sample holder according to aspect 26 or 27 wherein at least one indent of the first plate has a corresponding indent on the second plate.
   29. The personal sample holder according to any one of aspects 26 to 28 wherein at least one angular dimension about the pivot axis of a first tooth is different than that of a second tooth.
   30. The personal sample holder according to any one of aspects 26 to 28 wherein at least one angular dimension about the pivot axis of a first tooth and a second tooth are the same.
   31. The personal sample holder according to any one of aspects 26 to 30 wherein at least one angular dimension about the pivot axis of a first indent is different than that of a second indent.
   32. The personal sample holder according to any one of aspects 26 to 30 wherein at least one angular dimension about the pivot axis of a first indent and a second indent are the same.
   33. The personal sample holder according to any one of aspects 26 to 32 wherein the teeth and indents define discrete relative angular positions for at least one of the first and second arms about the pivot axis.
   34. The personal sample holder according to any one of aspects 26 to 33 wherein the first arm and the second arm each comprise at least one sidewall that extends in a direction that has at least one directional component that is parallel with the pivot axis.
   35. The personal sample holder according to aspect 34 wherein at least one of the sidewalls projects into one of the indents of the first plate or the second plate between two adjacent teeth to lock the pivot joint at one of the locking configurations.
   36. The personal sample holder according to any one of aspects 26 to 35 wherein the at least one arm is deformable to move to an adjacent indent upon the application of a sufficient force to the at least one arm.
   37. The personal sample holder according to any one of aspects 1 to 36 wherein the first arm or the second arm comprises a handle.
   38. The personal sample holder according to any one of aspects 1 to 37 wherein the sample collection vessel holder comprises a grip that comprises one or more raised portions.
   39. The personal sample holder according to any one of aspects 1 to 38 wherein the sample collection vessel holder or the second arm at the sample-collection location comprise one or more snap-together fittings that allow the sample collection vessel holder to attach to the second arm.

### Interpretation of Terms

Unless the context clearly requires otherwise, throughout the description and the claims:

“comprise”, “comprising”, and the like are to be construed in an inclusive sense, as opposed to an exclusive or exhaustive sense; that is to say, in the sense of “including, but not limited to”;

“connected”, “coupled”, or any variant thereof, means any connection or coupling, either direct or indirect, between two or more elements; the coupling or connection between the elements can be physical, logical, or a combination thereof;

“herein”, “above”, “below”, and words of similar import, when used to describe this specification, shall refer to this specification as a whole, and not to any particular portions of this specification;

“or”, in reference to a list of two or more items, covers all of the following interpretations of the word: any of the items in the list, all of the items in the list, and any combination of the items in the list;

the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Where a component (e.g. a plate, joint, arm, device, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A personal sample holder apparatus for aiding in the collection of personal samples, the personal sample holder apparatus comprising:
first and second elongated arms pivotally coupled to one another by a pivot joint to permit relative pivotal movement between the first and second arms about a pivot axis;
a sample collection vessel holder located at a sample-collection location of the second arm, the sample-collection location spaced apart from the pivot joint;
wherein the pivot joint is lockable at a plurality of discrete locking configurations to prevent relative pivotal movement between the first and second arms under application of forces associated with personal sample collection and each of the plurality of discrete locking configurations is at a corresponding relative angular orientation between the first and second arms.

2. The personal sample holder according to claim 1 wherein the pivot joint is configured to facilitate relative pivotal movement between locking configurations upon the application, to one or both of the first and second arms, of a force sufficient to deform one or both of the first and second arms.

3. The personal sample holder according to claim 1 wherein the pivot joint comprises a first plate, wherein there is physical interaction between the first plate and at least one of the first and second arms that locks the pivot joint in one of the locking configurations.

4. The personal sample holder according to claim 3 wherein the first plate comprises a plurality of teeth and a plurality of indents, wherein the indents provide concavities between the teeth, wherein the teeth protrude in a direction that has at least one directional component that is parallel with the pivot axis and the indents open in a direction that has at least one directional component that is parallel with the pivot axis.

5. The personal sample holder according to claim 4 wherein the teeth and indents define discrete relative angular positions of the first and second arms about the pivot axis.

6. The personal sample holder according to claim 4 wherein the first arm and the second arm each comprise at least one sidewall that extends in a direction that has at least one directional component that is parallel to the pivot axis, wherein at least one of the sidewalls projects into one of the indents between two adjacent teeth to lock the pivot joint at one of the locking configurations.

7. The personal sample holder according to claim 4 wherein at least one of the first and second arms is deformable to move to an adjacent indent upon the application of a sufficient force to at least one of the first arm and the second arm.

8. The personal sample holder according to claim 1 wherein the pivot joint comprises a first plate and a second plate, wherein physical interaction between at least one of the first and second arms and the first and second plates locks the pivot joint in one of the locking configurations.

9. The personal sample holder according to claim 8 wherein the first plate forms a snap-together fitting with the second plate.

10. The personal sample holder according to claim 9 wherein the first plate comprises:
a cylindrical bearing surface that extends in a direction that is parallel with the pivot axis, wherein the pivot axis extends through a cross-sectional center of the cylindrical bearing surface; and
one or more connecting protrusions located at locations radially spaced apart from the pivot axis and angularly spaced apart around the pivot axis, wherein the connecting protrusions extend from the cylindrical bearing surface in directions that have at least a directional component that is parallel with the pivot axis.

11. The personal sample holder according to claim 10 wherein the first plate comprises:
one or more resting protrusions located at locations radially spaced apart from the pivot axis, angularly spaced apart around the pivot axis and between pairs of connecting protrusions, wherein the resting protrusions extend from the cylindrical bearing surface in directions that have at least a directional component that is parallel with the pivot axis
wherein the resting protrusions extend, from the cylindrical bearing surface, for a shorter distance than the connecting protrusions.

12. The personal sample holder according to claim 10 wherein the connecting protrusions extend through openings on the second plate to secure the first plate to the second plate.

13. The personal sample holder according to claim 10 wherein the second arm comprises a bore-defining surface, wherein the cylindrical bearing surface bears against the bore-defining surface to facilitate relative pivotal motion between the first and second arms about the pivot axis.

14. The personal sample holder according to claim 8 wherein the first plate is integrally formed with the first arm.

15. The personal sample holder according to claim 8 wherein the first and second plates comprise a plurality of teeth and a plurality of indents, wherein the indents provide concavities between the teeth, wherein each tooth protrudes in a direction that has a directional component parallel with the pivot axis and each indent opens in a direction that has a directional component parallel with the pivot axis.

16. The personal sample holder according to claim 15 wherein at least one tooth of the first plate has a corresponding tooth on the second plate.

17. The personal sample holder according to claim 15 wherein the teeth and indents define discrete relative angular positions for at least one of the first and second arms about the pivot axis.

18. The personal sample holder according to claim 15 wherein the first arm and the second arm each comprise at least one sidewall that extends in a direction that has at least one directional component that is parallel with the pivot axis, wherein at least one of the sidewalls projects into one of the indents of the first plate or the second plate between two adjacent teeth to lock the pivot joint at one of the locking configurations.

19. The personal sample holder according to claim 15 wherein the at least one arm is deformable to move to an adjacent indent upon the application of a sufficient force to the at least one arm.

20. The personal sample holder according to claim 1 wherein the sample collection vessel holder or the second arm at the sample-collection location comprise one or more snap-together fittings that allow the sample collection vessel holder to attach to the second arm.
